(19) United States
(12) Patent Application Publication
(10) Pub. No.: US 2006/0205989 A1
(43) Pub. Date: Sep. 14, 2006

(54) Autothermal cracking process

(76) Inventors: Ian Raymond Little, Surrey (GB); Barry Martin Maunders, Surrey (GB); Brian Edward Messenger, Hampshire (GB)

Correspondence Address: NIXON & VANDERHYE, PC, 901 NORTH GLEBE ROAD, 11TH FLOOR, ARLINGTON, VA, 22203, US

(21) Appl. No.: 10/551,779

(22) PCT Filed: Mar. 24, 2004
(86) PCT No.: PCT/GB04/01278

§ 371 Date: Oct. 3, 2005

(30) Foreign Application Priority Data
Apr. 3, 2003 (GB) 0307759.1
Dec. 18, 2003 (GB) 0329346.1

(51) Int. Cl. C07C 4/02

(52) U.S. Cl. 585/652

(57) ABSTRACT

The present invention provides a process for the production of olefins which process comprises co-feeding at least one unsaturated hydrocarbon with a paraffinic hydrocarbon-containing feedstock and a molecular oxygen-containing gas to an autothermal cracker, wherein they are reacted in the presence of a catalyst capable of supporting combustion beyond the normal fuel rich limit of flammability to provide a hydrocarbon product stream comprising olefins.

The present invention relates to the production of mono-olefins by autothermal cracking of a paraffinic hydrocarbon having two or more carbon atoms especially autothermal cracking of ethane, propane, and butanes.

Olefins such as ethene and propene may be produced by a variety of processes including the steam cracking of hydrocarbons or by the dehydrogenation of paraffinic feedstocks. More recently, it has been disclosed that olefins may be produced by a process known as auto-thermal cracking. In such a process a paraffinic hydrocarbon feed is mixed with an oxygen-containing gas and contacted with a catalyst which is capable of supporting combustion beyond the normal fuel rich limit of flammability to provide a hydrocarbon product stream comprising olefins. The hydrocarbon feed is partially combusted and the heat produced is used to drive the dehydrogenation reaction. Such a process is described, for example, in EP-B1-0332289.

The steam cracking of hydrocarbons to produce mono-olefins normally co-produces other unsaturated hydrocarbons e.g. dienes and alkynes.

The dienes are usually separated from the steam cracker product stream which involves the use of large amounts of toxic flammable solvents e.g. acetonitrile. Once separated the dienes are considered high value products and are used in derivative processes e.g. elastomer production. However dienes are difficult to transport because they are readily degraded via oligomerisation and consequently derivative plants that employ diene feedstock are usually co-located with the sources of supply. Where there is no derivative capacity to use the dienes the production of dienes becomes problematic. This is because it is not desirable that dienes be recycled to a steam cracker due to their high propensity to cause carbonaceous fouling of the process equipment and therefore the dienes must be hydrogenated before being recycled to the steam cracker, or short furnace run-times must be tolerated, with consequent financial and operational disadvantages.

Similar problems arise with other unsaturated hydrocarbons produced by steam cracking, such as alkynes. These also have a high propensity to cause carbonaceous fouling if recycled to a steam cracker, and therefore must be hydrogenated before recycling, or short furnace run-times must be tolerated.

It has now been found that the autothermal cracking process can tolerate co-feeding unsaturated hydrocarbons without carbonaceous fouling, and therefore unsaturated hydrocarbons can be fed without causing reduced run-times. More particularly, it has now been found that the autothermal cracking process can be improved by co-feeding at least one unsaturated hydrocarbon, in particular a diene or alkyne, with the paraffinic hydrocarbon feed and the molecular oxygen-containing gas to the autothermal cracker. It has been found that co-feeding at least one unsaturated hydrocarbon can provide an increase in the olefin yield based on the amount of paraffinic hydrocarbon feed converted. Without wishing to be bound by theory, this is believed to be due to the propensity of co-fed unsaturated hydrocarbons to combust in preference to paraffinic hydrocarbons in the feed. Furthermore it has been found that the majority of the unsaturated hydrocarbon can be converted and, surprisingly, no significant carbon formation occurs on the catalyst, and unexpectedly low amounts of additional compounds e.g. benzene or toluene, associated with carbon formation on the catalyst are produced.

Accordingly the present invention provides a process for the production of olefins which process comprises feeding (i) a paraffinic hydrocarbon-containing feedstock; (ii) at least one unsaturated hydrocarbon and (iii) a molecular oxygen-containing gas to an autothermal cracker, wherein they are reacted in the presence of a catalyst capable of supporting combustion beyond the normal fuel rich limit of flammability to provide a hydrocarbon product stream comprising olefins.

“Unsaturated hydrocarbon”, as used herein, includes olefins.

Thus, the unsaturated hydrocarbon may be an alkene such as ethene, propene, butenes, pentenes, hexenes, heptenes, higher alkenes and cycloalkenes, such as cyclopropene, cyclobutene, cyclopentene(s), cyclohexene(s), cycloheptenes and higher cycloalkenes.

The unsaturated hydrocarbon may be an aromatic compound. Suitable aromatic compounds include benzene, toluene, xylenes, ethylbenzene, styrene and substituted styrenes, indene and substituted indenes. Where the autothermal cracker is operated at relatively low pressures, typically atmospheric pressure up to 5 barg, the preferred aromatic compounds are xylenes, indenes and styrenes. Where the autothermal cracker is operated at higher pressures, typically above 5 barg, the preferred aromatic compounds are benzene and/or toluene.

In a first preferred embodiment the unsaturated hydrocarbon is a diene. The diene(s) may be selected from any suitable dienes but are preferably selected from propadiene, 1, 2 butadiene, 1,3 butadiene, 1,3 pentadiene, 1,4 pentadiene, cyclopentadiene, 1,3 hexadiene, 1,4 hexadiene, 1,5 hexadiene, 2,4 hexadiene, 1,3 cyclohexadiene and 1,4 cyclohexadiene, and substituted derivatives of the above, e.g. alkyl substituted derivatives, e.g. methyl derivatives with more than one substitution per molecule, wherein the substituents may be the same or different. Most preferably the diene(s) are selected from 1,2 butadiene, 1,3 butadiene, 2 methyl 1,3 butadiene, 1,3 pentadiene, 1,4 pentadiene and cyclopentadiene. Advantageously the diene is 1,3 butadiene.

In a second preferred embodiment, the unsaturated hydrocarbon may be an alkyne such as acetylene, propyne and/or a butyne. A particularly preferred alkyne is acetylene.

A single unsaturated hydrocarbon or a mixture of unsaturated hydrocarbons may be fed to the autothermal cracker.

The process for the production of olefins according to the present invention produces predominantly mono-olefins (alkenes), especially ethene and propene, although quantities of other olefins may also be produced.

Although alkenes may be co-fed without carbonaceous fouling of the process, and may be expected to combust in preference to paraffinic hydrocarbons in the feed, it is generally preferred not to co-feed alkenes which are the same as the desired products of the process. However, co-feed of alkenes which are the same as the desired products of the process may take place if they are present as part of a stream also comprising other unsaturated hydrocarbons. Alternatively, for example, although it is generally preferred not to co-feed ethene and/or propene to an autothermal cracker for the production of predominantly ethene and/or propene, it may be advantageous to co-feed other alkenes, such as butenes, even if said process also produces said other alkenes.

In addition, co-feed of alkenes, such as ethene and propene, may also be advantageous where the alkene is present as unreacted alkene in an off-gas stream, which may also comprise alkane, of an alkene derivative process. Thus, ethene may be present in the off-gas of an ethene derivative process, such as a polyethylene process, an ethylbenzene process, an ethanol process and a vinyl acetate process. Propene may be present in the off-gas of a propene derivative process, such as a polypropylene process, an acrolein process, an iso-propanol process and an acrylic acid process.

Preferably, therefore, the unsaturated hydrocarbon fed to the autothermal cracker process of the present invention comprises at least one unsaturated hydrocarbon other than an alkene, such as at least one of a diene and an alkyne. More preferably, there is fed to the autothermal cracker at least one unsaturated hydrocarbon other than an alkene and less than 1 wt %, such as less than 0.5 wt %, of individual alkenes, such as ethene and propene, based on the weight of paraffinic hydrocarbon fed to the reactor. Even more preferably, there is fed to the autothermal cracker at least one unsaturated hydrocarbon other than an alkene and less than 1 wt %, such as less than 0.5 wt % of total alkenes, based on the weight of paraffinic hydrocarbon fed to the reactor. Most preferably, the feed to the autothermal cracker comprises at least one of a diene and an alkyne, and has a substantial absence of alkene.

In an alternative embodiment, the unsaturated hydrocarbon fed to the autothermal cracker process of the present invention may comprise at least one unsaturated hydrocarbon other than an aromatic compound.

The unsaturated hydrocarbon is provided as a separate feedstock than the paraffinic hydrocarbon-containing feedstock. However, it should be noted that the paraffinic hydrocarbon-containing feedstock may also contain unsaturated hydrocarbons, and the unsaturated hydrocarbon-containing feedstock may also contain paraffinic hydrocarbons.

The unsaturated hydrocarbon may derive from the product stream of a conventional steam cracking reactor. Alternatively the unsaturated hydrocarbon may derive from the off gas stream of a fluid catalytic cracking reactor or may derive from the off gas streams of a delayed coker unit, a visbreaker unit or an alkylation unit. The unsaturated hydrocarbon may also be provided as a refinery stream derived from a coker, fluid catalytic cracking (FCC) or residue catalytic cracking (RCC) units.

In addition the unsaturated hydrocarbon may be provided by a plastics recycling process e.g. pyrolytic polymer cracking.

In one embodiment of the present invention the unsaturated hydrocarbon is provided as a portion of the product stream from a polymer cracking reactor. As well as unsaturated hydrocarbons, the product stream from the polymer cracking reactor may also comprise paraffinic hydrocarbons and, hence, may also provide at least a portion of the total paraffinic hydrocarbon fed to the process of the present invention.

The autothermal cracking reactor produces a product stream comprising unsaturated hydrocarbons (olefins and other unsaturated hydrocarbons). In a preferred embodiment of the invention the unsaturated hydrocarbon fed to the autothermal cracking reactor derives from the autothermal cracking product stream.

Consequently the present invention also provides a process for the production of olefins which process comprises the steps of:

(a) feeding a paraffinic hydrocarbon-containing feedstock and a molecular oxygen-containing gas to an autothermal cracker wherein they are reacted in the presence of a catalyst capable of supporting combustion beyond the normal fuel rich limit of flammability to provide a hydrocarbon product stream comprising olefins

(b) recovering at least a portion of the olefins produced in step (a) and

(c) recycling at least one unsaturated hydrocarbon produced in step (a) back to the autothermal cracker.

In one preferred embodiment, the hydrocarbon product stream produced in step (a) is separated into a first stream comprising hydrocarbons containing less than 4 carbon atoms and a second stream comprising hydrocarbons containing at least 4 carbon atoms.

Consequently a further embodiment of the invention provides a process for the production of ethene and/or propene which process comprises the steps of:

(a) feeding a paraffinic hydrocarbon-containing feedstock and a molecular oxygen-containing gas to an autothermal cracker wherein they are reacted in the presence of a catalyst capable of supporting combustion beyond the normal fuel rich limit of flammability to provide a hydrocarbon product stream comprising ethene and/or propene

(b) separating the hydrocarbon product stream produced in step (a) into a first stream comprising hydrocarbons containing less than 4 carbon atoms and a second stream comprising hydrocarbons containing at least 4 carbon atoms, including at least one unsaturated hydrocarbon containing at least 4 carbon atoms

(c) recovering ethene and/or propene from the first stream and

(d) recycling at least a portion of the second stream to the autothermal cracker.

In this embodiment, preferably the unsaturated hydrocarbon containing at least 4 carbon atoms is recovered from the second stream and recycled to the autothermal cracker.

The unsaturated hydrocarbon containing at least 4 carbon atoms may be any unsaturated compound as herein described above containing at least 4 carbon atoms. Preferably the unsaturated hydrocarbon containing at least 4 carbon atoms is selected from 1,2 butadiene, 1, 3 butadiene, 2 methyl 1,3 butadiene, 1,3 pentadiene, 1,4 pentadiene and cyclopentadiene and is advantageously 1, 3 butadiene.

As stated above, in a second preferred embodiment the unsaturated hydrocarbon may be an alkyne such as acetylene, propyne and/or a butyne.

Consequently, the present invention also provides a process for the production of ethene and/or propene which process comprises the steps of:

(a) feeding a paraffinic hydrocarbon-containing feedstock and a molecular oxygen-containing gas to an autothermal cracker wherein they are reacted in the presence of a catalyst capable of supporting combustion beyond the normal fuel rich limit of flammability; to provide a hydrocarbon product stream comprising ethene and/or propene, and at least one alkyne

(b) recovering at least a portion of the ethene and/or propene produced in step (a) and

(c) recycling at least a portion of the at least one alkyne produced in step (a) back to the autothermal cracker.

In this preferred embodiment, it has been found that co-feeding at least one alkyne can provide significant improvements in ethene yield and, in addition, that co-feeding alkynes can suppress methane yield.

A single alkyne or a mixture of alkynes may be passed to the autothermal cracker. Alternatively, a mixture of one or more alkynes with one or more other unsaturated compounds, such as one or more alkenes and/or dienes, may be passed to the autothermal cracker.

As stated above, at least a portion of the unsaturated hydrocarbon derives from the autothermal cracking product stream itself i.e. from the hydrocarbon product stream. If required, the unsaturated hydrocarbon derived from the hydrocarbon product stream may be supplemented by additional unsaturated hydrocarbon from one or more other sources, such as from the product stream of a conventional steam cracking reactor, the off gas stream of a fluid catalytic cracking reactor, the off gas streams of a delayed coker unit, a visbreaker unit or an alkylation unit or from a plastics recycling process e.g. pyrolytic polymer cracking.

Where the unsaturated hydrocarbon is an alkyne (or mixture comprising at least one alkyne), a particularly preferred source of supplemental alkyne, where required, is acetylene obtained by acetylene generation from methane. Such acetylene generation processes are well-known, and include, for example, oxidative and non-oxidative pyrolysis and oxidative coupling processes. Most preferably, the methane for the acetylene generation may itself be derived from the autothermal cracking product stream, giving an overall process in which at least some of any methane formed in the autothermal cracking process is converted to acetylene, which is then co-fed back to the autothermal cracking process to improve olefin yield and suppress formation of further methane. Hence, when the unsaturated hydrocarbon is an alkyne the present process can provide significant benefit (i.e. reduction) in the overall selectivity to methane.

The paraffinic hydrocarbon-containing feedstock may suitably be ethane, propane or butane, or a mixture thereof. The hydrocarbon-containing feedstock may comprise other hydrocarbons and optionally other materials, for example, nitrogen, carbon monoxide, carbon dioxide, steam or hydrogen. In particular, the paraffinic hydrocarbon-containing feedstock may also contain unsaturated hydrocarbons, such as olefins and aromatics, in addition to the at least one unsaturated hydrocarbon feedstock. The paraffinic hydrocarbon-containing feedstock may contain a fraction such as naphtha, gas oil, vacuum gas oil, or mixtures thereof. Usually the paraffinic hydrocarbon-containing feedstock comprises a mixture of gaseous paraffinic hydrocarbons, principally comprising ethane, resulting from the separation of methane from natural gas.

The paraffinic hydrocarbon-containing feedstock, the at least one unsaturated hydrocarbon and the molecular oxygen-containing gas may all be passed as separate streams to the autothermal cracker.

Usually the at least one unsaturated hydrocarbon is pre-mixed with the paraffinic hydrocarbon-containing feedstock and subsequently passed to the autothermal cracker. The resultant stream usually has the unsaturated hydrocarbon at a weight percentage of at least 0.01 wt %, preferably at least 0.1 wt %, most preferably at least 1 wt % and advantageously at least 2 wt % based on the weight of paraffinic hydrocarbon.

Usually the unsaturated hydrocarbon has a weight percentage of between 0.01-50 wt %, preferably between 0.1-30 wt %, most preferably between 1-20 wt % and advantageously between 2-15 wt % based on the weight of the paraffinic hydrocarbon.

Where the unsaturated hydrocarbon is a diene (or mixture comprising at least one diene), the unsaturated hydrocarbon preferably has a weight percentage of between 1-20 wt % of diene, preferably between 2-15 wt % of diene, based on the weight of the paraffinic hydrocarbon.

Where the unsaturated hydrocarbon is an alkyne (or mixture comprising at least one alkyne), the unsaturated hydrocarbon preferably has a weight percentage of between 0.1-5 wt % of alkyne, preferably between 1-5 wt % of alkyne, based on the weight of the paraffinic hydrocarbon.

The molecular oxygen-containing gas may suitably be either oxygen or air. Preferably the molecular oxygen-containing gas is oxygen, optionally diluted with an inert gas, for example nitrogen.

The ratio of paraffinic hydrocarbon-containing feedstock to molecular oxygen-containing gas mixture is suitably from 5 to 13.5 times the stoichiometric ratio of hydrocarbon to oxygen-containing gas for complete combustion to carbon dioxide and water. The preferred ratio is from 5 to 9 times the stoichiometric ratio of hydrocarbon to oxygen-containing gas.

Additional feed streams comprising at least one from carbon monoxide, carbon dioxide, steam and hydrogen may also be passed to the autothermal cracker. Preferably an additional feed stream comprising hydrogen is passed to the autothermal cracker. Preferably the additional feed stream comprising hydrogen is pre-mixed with the paraffinic hydrocarbon-containing feedstock and subsequently passed to the autothermal cracker.

The autothermal cracker may suitably be operated at a temperature greater than 500° C., for example greater than 650° C., typically greater than 750° C., and preferably greater than 800° C. The upper temperature limit may suitably be up to 1200° C., for example up to 1100° C., preferably up to 1000° C.

In general, the autothermal cracker may be operated at atmospheric or elevated pressure. Pressures of 1-40 barg may be suitable, preferably a pressure of 1-5 barg e.g. 1.8 barg is employed. However a total pressure of greater than 5 barg may be used, usually a total pressure of greater than 15 barg. Advantageously the autothermal cracker is operated in a pressure range of between 15-40 barg, such as between 20-30 barg e.g. 25 barg.

Where the unsaturated hydrocarbon is an alkene, an aromatic compound or a mixture of alkenes and/or aromatic compounds, the autothermal cracker is preferably operated at a total pressure of greater than 5 barg, usually a total pressure of greater than 15 barg, and advantageously in a pressure range of between 1540 barg, such as between 20-30 barg e.g. 25 barg.

Preferably, the paraffinic hydrocarbon-containing feedstock, the gas comprising at least one unsaturated hydrocarbon and the molecular oxygen-containing gas are fed to the autothermal cracker in admixture under a Gas Hourly Space Velocity (GHSV) of greater than 80,000 hr⁻¹. Preferably, the GHSV exceeds 200,000 hr⁻¹, especially greater than 1,000,000 hr⁻¹. For the purposes of the present invention GHSV is defined as:—. (volume of total feed at NTP hour)/(volume of catalyst bed).

Suitably the catalyst is a supported platinum group metal. Preferably, the metal is either platinum or palladium, or a mixture thereof. Where the unsaturated hydrocarbon is an alkyne (or mixture comprising at least one alkyne), the metal preferably comprises a mixture of platinum and palladium

Although a wide range of support materials is available, it is preferred to use alumina as the support. The support material may be in the form of spheres, other granular shapes or ceramic foams. Preferably, the foam is a monolith which is a continuous multichannel ceramic structure, frequently of a honeycomb appearance. A preferred support for the catalytically active metals is a gamma alumina. The support is loaded with platinum and/or palladium by conventional methods well known to those skilled in the art. Advantageously catalyst promoters may also be loaded onto the support. Suitable promoters include copper and tin. Usually the products are quenched as they emerge from the autothermal cracker such that the temperature is reduced to less than 650° C. within less than 150 milliseconds of formation.

Where the pressure of the autothermal cracker is maintained at a pressure of between 1.5-2.0 barg usually the products are quenched and the temperature reduced to less than 650° C. within 100-150 milliseconds of formation.

Where the pressure of the autothermal cracker is maintained at a pressure of between 2.0-5.0 barg usually the products are quenched and the temperature reduced to less than 650° C. within 50-100 milliseconds of formation.

Where the pressure of the autothermal cracker is maintained at a pressure of between 5.0-10.0 barg usually the products are quenched and the temperature reduced to less than 650° C. within less than 50 milliseconds of formation.

Where the pressure of the autothermal cracker is maintained at a pressure of between 10.0-20.0 barg usually the products are quenched and the temperature reduced to less than 650° C. within 20 milliseconds of formation.

Finally where the pressure of the autothermal cracker is maintained at a pressure of greater than 20.0 barg usually the products are quenched and the temperature reduced to less than 650° C. within 10 milliseconds of formation.

This avoids further reactions taking place and maintains a high olefin selectivity.

The products may be quenched using rapid heat exchangers of the type familiar in steam cracking technology. Additionally or alternatively, a direct quench may be employed. Suitable quenching fluids include water.

The present invention usually provides a percentage conversion of gaseous paraffinic hydrocarbon of greater than 40%, preferably greater than 50%, and most preferably greater than 60%.

Furthermore the present invention usually provides a selectivity towards mono-olefins of greater than 50%, preferably greater than 60%, and most preferably greater than 70%.

In a further aspect of the present invention, there is provided a process for the production of olefins Which process comprises feeding a paraffinic hydrocarbon, at least one unsaturated hydrocarbon and a molecular oxygen-containing gas to an autothermal cracker wherein they are reacted in the presence of a catalyst capable of supporting combustion beyond the normal fuel rich limit of flammability to provide a hydrocarbon product stream comprising olefins, said process being characterised in that the total hydrocarbon fed to the autothermal cracker comprises at least 20 wt % of unsaturated hydrocarbons.

In this aspect of the present invention, both the paraffinic hydrocarbon and the at least one unsaturated hydrocarbon may be provided as a single hydrocarbon-containing feedstock comprising at least 20 wt % of unsaturated hydrocarbons. For example, the single hydrocarbon-containing feedstock may be a stream boiling in the middle distillate range (typically 150° C. to 400° C.) or in the naphtha-range (typically 30° C. to 220° C.), but with significantly higher unsaturated hydrocarbon content than would conventionally be fed to a steam cracker (without considerable dilution by saturated feeds from other sources). Suitable feedstocks include refinery streams derived from coker, fluid catalytic cracking (FCC) or residue catalytic cracking (RCC) units.

Because of the ability of the autothermal cracker to tolerate significant quantities of unsaturated hydrocarbons without carbonaceous fouling, streams which would not conventionally be considered for steam cracking (without considerable dilution by saturated feeds from other sources) can be readily fed to the autothermal cracker. The removal of constraint on the unsaturated hydrocarbon content of the hydrocarbon-containing feedstock, may also allow processes which conventionally generate cracking feedstocks, such as crude oil distillation to produce straight-run naphtha, to be operated more advantageously.

Preferably the total hydrocarbon fed to the autothermal cracker comprises 20 to 70 wt %, such as 25 to 50 wt %, of unsaturated hydrocarbons. Typically, the unsaturated hydrocarbons may comprise olefins, such as at least 10 wt % olefins, and aromatics, such as at least 10 wt % aromatics. As used in this aspect, the percent by weight (wt %) is based on the total weight of hydrocarbons in the combined feeds to the autothermal cracker.

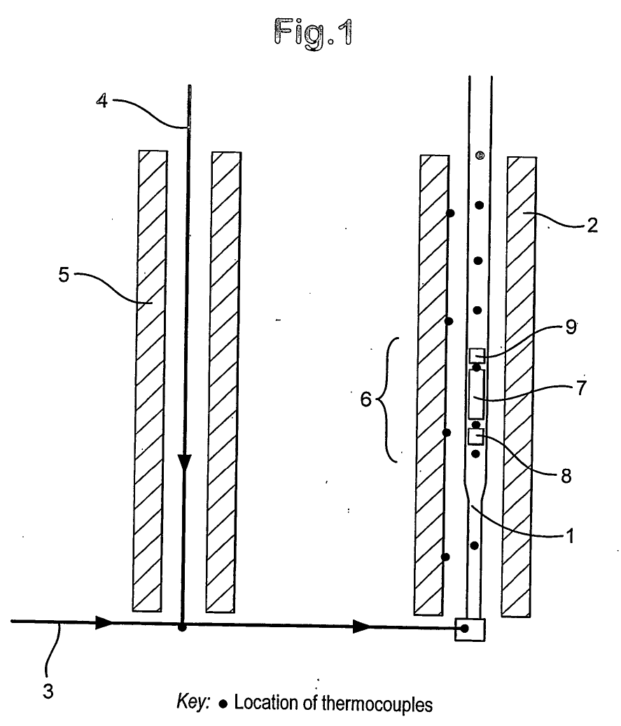

The invention will now be illustrated in the following examples and FIG. 1.

FIG. 1 represents a schematic view of an autothermal cracking apparatus.

FIG. 1 depicts an autothermal cracking apparatus comprising a quartz reactor, 1, surrounded by an electrically-heated furnace, 2. The reactor, 1, is coupled to an oxygen-containing gas supply, 3, and a hydrocarbon feed supply, 4 (for both the paraffinic hydrocarbon and unsaturated hydrocarbon). The hydrocarbon feed supply, 4, is pre-heated in an electrically heated furnace, 5. Optionally, the hydrocarbon feed may comprise a further co-feed such as hydrogen and a diluent such as nitrogen. In use, the reactor, 1, is provided with a catalyst zone, 6, which is capable of supporting combustion beyond the fuel rich limit of flammability and comprises a catalyst bed, 7. The catalyst bed, 7, is positioned between heat shields, 8, 9.

In use, the furnace, 2, is set so as to minimise heat losses. As the reactants contact the catalyst bed, 7, some of the hydrocarbon feed combusts to produce water and carbon oxides. The optional hydrogen co-feed also combusts to produce water. Both of these combustion reactions are exothermic, and the heat produced therefrom is used to drive the cracking of the hydrocarbon to produce olefin.

### EXAMPLES

Catalyst A

An auto-thermal cracking catalyst comprising 3 wt % platinum and 1 wt % copper deposited on an alumina foam (15 mm diameter×30 mm deep, 30 pores per inch supplied by Vesuvius Hi-Tech Ceramics, Alfred, N.Y. USA) was prepared by repeated impregnation with solutions of tetraamineplatinum (II) chloride and copper (II) chloride in deionised water. The metal salt solutions were of sufficient concentration to achieve the desired loadings of Pt and Cu if all the metal salt were incorporated into the final catalyst formulation. After each impregnation, any excess solution was removed, and the alumina foam was dried in air at 120° C.-140° C. and calcined in air at 450° C. before the next impregnation. Once all the solution had been adsorbed, the foams were dried and reduced under hydrogen/nitrogen atmosphere at 650-700° C. for 1 hour.

Catalyst B

An auto-thermal cracking catalyst comprising platinum and palladium deposited on alumina spheres was prepared by impregnation, using incipient wetness, of 100 g of alumina spheres (supplied by Condea, 1.8 mm diam. alumina spheres, Surface Area 210 m²/g), with a solution containing 4.415 g of tetraamineplatinum (II) chloride and 0.495 g of tetraaminepalladium (II) chloride in deionised water. The spheres were dried at 120° C. for 1 hour and then calcined, in air, at 1200° C. for 6 hours.

### Example 1

The auto-thermal cracking catalyst comprising platinum and copper deposited on alumina foam (two blocks of Catalyst A resulting in a bed 60 mm deep) was placed in the autothermal cracker and the cracker was heated to 850° C.

A feed stream comprising ethane, nitrogen and hydrogen was passed to the autothermal cracker. Oxygen was then passed to the autothermal cracker to initiate the reaction. The hydrogen to oxygen volume ratio was maintained at 1.9:1 (v/v). The reaction was performed at atmospheric pressure.

Samples were analysed at oxygen to ethane feed ratios of 0.35, 0.44, 0.53 and 0.61 (v/v).

The nitrogen was then replaced with a feed stream comprising 9.65 volume % of 1,3 butadiene in nitrogen and the analysis repeated.

The % conversion of ethane and the selectivity towards ethylene was measured and the results are shown in table 1.

### Example 2

Example 1 was repeated using a hydrogen to oxygen volume ratio of 1:1 (v/y). The % conversion of ethane and the selectivity towards ethylene was measured and the results are shown in table 2.

### Example 3

Example 1 was repeated using a hydrogen to oxygen volume ratio of 0.5:1 (v/v)

The samples were taken at oxygen to ethane feed ratios of 0.35, 0.44, and 0.53 (v/v). The % conversion of ethane and the selectivity towards ethylene was measured and the results are shown in table 3.

TABLE1
Autothermal cracking of ethane and ethane with butadiene over a Pt—Cu
catalyst with a hydrogen to oxygen volume ratio of 1.9:1 (v/v).

| | | ethane | | ethane | | ethane | | ethane |
|---|---|---|---|---|---|---|---|---|
| | ethane | butadiene | ethane | butadiene | ethane | butadiene | ethane | butadiene |
| Total feed rate nl/min | 9.03 | 9.02 | 9.19 | 9.18 | 9.18 | 9.17 | 9.16 | 9.11 |
| O2:C2H6 (v/v) | 0.353 | 0.353 | 0.435 | 0.435 | 0.527 | 0.527 | 0.605 | 0.605 |
| H2:O2 (v/v) | 1.994 | 1.986 | 1.958 | 1.969 | 1.868 | 1.886 | 1.841 | 1.826 |
| N2:O2 (v/v) | 0.543 | 0.490 | 0.492 | 0.433 | 0.446 | 0.387 | 0.426 | 0.375 |
| 1,3-butadiene:ethane | — | 0.020 | — | 0.022 | — | 0.024 | — | 0.027 |
| (v/v) | | | | | | | | |
| Ethane | 46.00 | 42.87 | 58.74 | 55.88 | 74.76 | 72.19 | 84.65 | 82.40 |
| conversion (%) | | | | | | | | |
| Oxygen | 98.42 | 98.12 | 98.30 | 98.42 | 98.48 | 98.75 | 98.73 | 98.84 |
| conversion (%) | | | | | | | | |
| Butadiene | — | 93.05 | — | 96.24 | — | 97.66 | — | 100.00 |
| Conversion (%) | | | | | | | | |
| Ethene yield | 36.50 | 35.59 | 44.93 | 44.87 | 53.16 | 54.13 | 55.87 | 57.93 |
| (g per 100 g | | | | | | | | |
| ethane feed) | | | | | | | | |
| Aromatics yield | 0.03 | 0.08 | 0.03 | 0.04 | 0.14 | 0.02 | 0.22 | 0.02 |
| (g per 100 g | | | | | | | | |
| ethane feed) | | | | | | | | |
| Ethene selectivity | 79.35 | 83.03 | 76.49 | 80.29 | 71.11 | 74.98 | 66.00 | 70.30 |
| (g per 100 g ethane | | | | | | | | |
| converted) | | | | | | | | |

TABLE 2
Autothermal cracking of ethane and ethane with butadiene over a Pt—Cu
catalyst with a hydrogen to oxygen volume ratio of 1:1 (v/v).

| | | ethane | | ethane | | ethane | | ethane |
|---|---|---|---|---|---|---|---|---|
| | ethane | butadiene | Ethane | butadiene | ethane | butadiene | ethane | butadiene |
| Total feed rate (nl/min) | 7.62 | 7.62 | 7.77 | 7.76 | 7.76 | 7.74 | 7.71 | 7.66 |
| O2:C2H6 (v/v) | 0.353 | 0.353 | 0.435 | 0.435 | 0.527 | 0.527 | 0.602 | 0.602 |
| H2:O2 (v/v) | 0.996 | 0.994 | 1.069 | 1.068 | 1.055 | 1.048 | 1.058 | 1.046 |
| N2:O2 (v/v) | 0.538 | 0.484 | 0.483 | 0.433 | 0.443 | 0.400 | 0.431 | 0.378 |
| 1,3-butadiene:ethane | — | 0.021 | — | 0.023 | — | 0.026 | — | 0.028 |
| (v/v) | | | | | | | | |
| ethane | 47.86 | 46.15 | 60.97 | 59.14 | 77.10 | 75.43 | 86.44 | 84.98 |
| conversion (%) | | | | | | | | |
| oxygen | 98.80 | 98.69 | 98.85 | 98.74 | 98.82 | 98.79 | 98.94 | 98.97 |
| conversion (%) | | | | | | | | |
| butadiene | — | 92.86 | — | 92.01 | — | 97.47 | — | 98.37 |
| conversion (%) | | | | | | | | |
| Ethene yield | 35.59 | 36.14 | 44.10 | 44.98 | 52.13 | 53.58 | 54.35 | 56.33 |
| (g/100 g ethane feed) | | | | | | | | |
| Aromatics yield | 0.03 | 0.03 | 0.02 | 0.05 | 0.03 | 0.13 | 0.10 | 0.13 |
| (g per 100 g | | | | | | | | |
| ethane feed) | | | | | | | | |
| Ethene selectivity | 74.36 | 78.31 | 72.33 | 76.06 | 67.61 | 71.03 | 62.87 | 66.29 |
| (g per 100 g ethane | | | | | | | | |
| converted) | | | | | | | | |

TABLE 3
Autothermal cracking of ethane and ethane with butadiene over a Pt—Cu
catalyst with a hydrogen to oxygen volume ratio of 0.5:1 (v/v).

| | | ethane | | ethane | | ethane |
|---|---|---|---|---|---|---|
| | ethane | butadiene | ethane | butadiene | ethane | butadiene |
| total feed rate nl/min | 6.95 | 6.95 | 6.76 | 6.74 | 6.62 | 6.58 |
| O2:C2H6 (v/v) | 0.435 | 0.435 | 0.527 | 0.527 | 0.605 | 0.605 |
| H2:O2 (v/v) | 0.546 | 0.542 | 0.474 | 0.477 | 0.460 | 0.441 |
| N2:O2 (v/v) | 0.488 | 0.442 | 0.452 | 0.399 | 0.425 | 0.378 |
| 1,3-butadiene:ethane | — | 0.024 | — | 0.025 | — | 0.030 |
| (v/v) | | | | | | |
| ethane conversion (%) | 64.01 | 63.17 | 78.83 | 79.02 | 88.11 | 87.97 |
| oxygen conversion (%) | 98.43 | 98.42 | 98.63 | 98.64 | 98.86 | 98.85 |
| butadiene conversion (%) | — | 98.59 | — | 95.49 | — | 93.80 |
| Ethene yield | 43.84 | 45.62 | 50.63 | 51.80 | 51.68 | 53.03 |
| (g per 100 g ethane feed) | | | | | | |
| Aromatics yield | 0.01 | 0.05 | 0.08 | 0.08 | 0.23 | 0.34 |
| (g per 100 g ethane feed) | | | | | | |
| Ethene selectivity (g per | 68.49 | 72.21 | 64.23 | 65.56 | 58.66 | 60.29 |
| 100 g ethane converted) | | | | | | |

It can be seen from all the above examples that the ethene yield is generally increased and that in all cases with the addition of butadiene the ethene selectivity is increased. Furthermore it can also be seen that the addition of the butadiene does not result in any significant carbon formation on the catalyst surface due to the fact that only low amounts of aromatics are produced.

### Example 4

The auto-thermal cracking catalyst comprising platinum and palladium deposited on alumina spheres (Catalyst B) was placed in the autothermal cracker and the cracker was heated to 850° C. Catalyst bed dimensions were 15 mm diameter by 60 mm deep

A feed stream comprising ethane, nitrogen and hydrogen was passed to the autothermal cracker. Oxygen was then passed to the autothermal cracker to initiate the reaction. The hydrogen to oxygen volume ratio was maintained at 0.7:1 (v/v). The reaction was performed at atmospheric pressure.

Samples were analysed at three oxygen:hydrocarbon feed ratios in the range 0.51-0.60 wt/wt.

Acetylene was then added at a level of 2.5 vol % of acetylene in ethane and the analyses repeated.

The % conversion of ethane and the selectivity towards ethylene was measured and the results are shown in table 4 for the data at O2:hydrocarbon (ethane plus acetylene) weight ratios of ca. 0.51, 0.56 and 0.60.

It can be seen from Table 4 that the ethene yield and selectivity are both increased with the addition of acetylene. Furthermore it can also be seen that the addition of the acetylene does not result in any significant carbon formation on the catalyst surface due to the fact that only low amounts of aromatics are produced.

In addition, and surprisingly, methane yield is observed to fall on addition of acetylene. It might be expected that the methane yield would increase since methane is a secondary product of the dehydrogenation/cracking reaction of ethane to produce ethylene. Thus, the presence of acetylene appears to inhibit methane formation.

TABLE 4
Autothermal cracking of ethane and ethane with acetylene over a Pt—Pd
catalyst with an hydrogen to oxygen volume ratio of 0.7:1 (v/v).

| | | ethane | | ethane | | ethane |
|---|---|---|---|---|---|---|
| | | plus | | plus | | plus |
| | Ethane | acetylene | ethane | acetylene | ethane | acetylene |
| total feed nl/min | 6.02 | 6.00 | 5.98 | 5.96 | 5.91 | 5.94 |
| O2/C2H6 (v/v) | 0.488 | 0.502 | 0.526 | 0.541 | 0.563 | 0.571 |
| H2:O2 (v/v) | 0.701 | 0.701 | 0.699 | 0.699 | 0.697 | 0.697 |
| N2:O2 (v/v) | 0.598 | 0.592 | 0.582 | 0.574 | 0.565 | 0.566 |
| acetylene:ethane (v/v) | — | 0.026 | — | 0.026 | — | 0.026 |
| O2/hydrocarbon (wt/wt) | 0.520 | 0.524 | 0.561 | 0.565 | 0.600 | 0.595 |
| ethane conversion (%) | 67.5 | 73.1 | 73.8 | 78.5 | 79.1 | 82.2 |
| oxygen conversion (%) | 99.4 | 99.3 | 99.4 | 99.4 | 99.3 | 99.5 |
| Ethene yield | 45.17 | 50.41 | 48.13 | 52.62 | 49.77 | 53.68 |
| (g per 100 g ethane feed) | | | | | | |
| Methane yield | 4.02 | 3.96 | 4.90 | 4.74 | 5.75 | 5.37 |
| (g per 100 g ethane feed) | | | | | | |
| Aromatics yield | 0.000 | 0.009 | 0.000 | 0.010 | 0.003 | 0.014 |
| (g per 100 g ethane feed) | | | | | | |
| Ethene selectivity (g per | 67.0 | 69.0 | 65.2 | 67.1 | 62.9 | 65.3 |
| 100 g ethane converted) | | | | | | |

### Example 5

An auto-thermal cracking catalyst comprising platinum (two blocks of catalyst comprising 3 wt % platinum) was placed in the autothermal cracker and the cracker was heated to 800° C.

A feed stream comprising n-pentane, nitrogen and hydrogen was passed to the autothermal cracker. Oxygen was then passed to the autothermal cracker to initiate the reaction. The hydrogen to oxygen volume ratio was maintained at 0.5:1 (v/v). The reaction was performed at atmospheric pressure.

Samples were analysed at oxygen to pentane feed ratios of 0.752, 0.675 and 0.636. (v/v).

An aromatic containing feedstream comprising xylene and indene at a weight ratio of 4:1 xylene:indene was then introduced to give a total aromatics to n-pentane ratio of 0.078 wt/wt.

The % conversion of n-pentane and the selectivity towards ethene was measured and the results are shown in table 5.

It can be seen from Table 5 that the ethene yield and selectivity are both increased with the addition of aromatic compounds.

TABLE 5
Autothermal cracking of n-pentane and n-pentane with aromatics over
a Pt catalyst with a hydrogen to oxygen volume ratio of 0.5:1 (v/v).

| | | pentane | | pentane | | pentane |
|---|---|---|---|---|---|---|
| | pentane | aromatic | pentane | aromatic | pentane | aromatic |
| total feed rate nl/min | 3.33 | 3.21 | 3.17 | 3.12 | 3.08 | 3.00 |
| O2/C5H12 (v/v) | 0.752 | 0.898 | 0.675 | 0.853 | 0.636 | 0.786 |
| H2:O2 (v/v) | 0.504 | 0.500 | 0.505 | 0.500 | 0.505 | 0.505 |
| N2:O2 (v/v) | 0.383 | 0.335 | 0.445 | 0.346 | 0.472 | 0.384 |
| aromatic:pentane (wt/wt) | — | 0.078 | — | 0.078 | — | 0.078 |
| pentane conversion (%) | 84.21 | 83.06 | 79.60 | 79.60 | 76.56 | 77.16 |
| oxygen conversion (%) | 98.17 | 97.74 | 98.28 | 97.80 | 98.37 | 97.90 |
| aromatics conversion (%) | — | 66.97 | — | 62.56 | — | 60.81 |
| Ethene yield | 33.49 | 33.53 | 30.47 | 31.52 | 28.68 | 29.86 |
| (g per 100 g pentane feed) | | | | | | |
| Ethene selectivity (g per | 39.77 | 40.37 | 38.28 | 39.59 | 37.46 | 38.70 |
| 100 g pentane converted) | | | | | | |

1-14. (canceled)

15. A process for the production of olefins which process comprises feeding a paraffinic hydrocarbon-containing feedstock and a molecular oxyen-containing gas to an autothermal cracker, wherein the paraffinic hydrocarbon is partially combusted in the presence of a catalyst capable of supporting combustion beyond the normal fuel rich limit of flammability and the heat produced drives dehydrogenation to provide a hydrocarbon product stream comprising olefins, characterised in that
a) at least one unsaturated hydrocarbon is provided as a separate feedstock to the paraffinic hydrocarbon-containing feedstock and is co-fed to the autothermal cracker, and
b) the unsaturated hydrocarbon has a weight percentage of between 1-20 wt % based on the weight of paraffinic hydrocarbon.

16. A process according to claim 15, wherein the unsaturated hydrocarbon is one or more of an alkene, an aromatic compound, a diene and an alkyne.

17. A process according to claim 16, wherein the unsaturated hydrocarbon is 1,2 butadiene, 1,3 butadiene, 2 methyl 1,3 butadiene, 1,3 pentadiene, 1,4 pentadiene and/or cyclopentadiene, preferably 1,3 butadiene.

18. A process according to claim 16, wherein the unsaturated hydrocarbon is acetylene, propyne and/or a butyne, preferably acetylene.

19. A process according to claim 16, wherein the autothermal cracker is operated at a total pressure of greater than 5 barg and the unsaturated hydrocarbon is benezene and/or toluene.

20. A process according to claim 15, wherein the unsaturated hydrocarbon fed to the autothermal cracker comprises at least one unsaturated hydrocarbon other than an alkene, such as at least one of a diene and an alkyne, and less than 1 wt %, such as less than 0.5 wt %, of total alkenes, based on the weight of paraffinic hydrocarbon fed to the reactor.

21. A process according to claim 15, wherein the unsaturated hydrocarbon derives from the product stream of a steam cracking reactor, the off gas stream of a fluid catalytic cracking reactor, the off gas streams of a delayed coker unit, a visbreaker unit or an alkylation unit or from a plastics recycling process, such as pyrolytic polymer cracking.

22. A process according to claim 15, wherein the unsaturated hydrocarbon fed to the autothermal cracking reactor derives from the autothermal cracking product stream.

23. A process according to claim 22, which process comprises the steps of:
(a) feeding a paraffinic hydrocarbon-containing feedstock and a molecular oxygen-containing gas to an autothermal cracker wherein they are reacted in the presence of a catalyst capable of supporting combustion beyond the normal fuel rich limit of flammability to provide a hydrocarbon product stream comprising olefins
(b) recovering at least a portion of the olefins product in step (a) and
(c) recycling at least one unsaturated hydrocarbon product in step (a) back to the autothermal cracker.

24. A process according to claim 23 which process comprises the steps of:
(a) feeding a paraffinic hydrocarbon-containing feedstock and a molecular oxygen-containing gas to an autothermal cracker wherein they are reacted in the presence of a catalyst capable of supporting combustion beyond the normal fuel rich limit of flammability to provide a hydrocarbon product stream comprising ethane and/or propene
(b) separating the hydrocarbon product stream product in step (a) into a first stream comprising hydrocarbons containing less than 4 carbon atoms and a second stream comprising hydrocarbons containing at least 4 carbon atoms, including at least one unsaturated hydrocarbon containing at least 4 carbon atoms
(c) recovering ethane and/or propene from the first stream and
(d) recycling at least a portion of the second stream to the autothermal cracker.

25. A process according to claim 24 wherein the unsaturated hydrocarbon containing at least 4 carbon atoms is selected from 1,2 butadiene, 1,3 butadiene, 2 methyl 1,3 butadiene, 1,3 pentadiene, 1,4 pentadiene and cyclopentadiene, and preferably is 1,3 butadiene.

26. A process according to claim 23 which process comprises the steps of:
(a) feeding a paraffinic hydrocarbon-containing feedstock and a molecular oxygen-containing gas to an autothermal cracker wherein they are reacted in the presence of a catalyst capable of supporting combustion beyond the normal fuel rich limit of flammability to provide a hydrocarbon product stream comprising ethane and/or propene, and at least one alkyne
(b) recovering at least a portion of the ethane and/or propene product in step (a) and
(c) recycling at least a portion of the at least one alkyne product in step (a) back to the autothermal cracker.

27. A process for the production of olefins which process comprises feeding a paraffinic hydrocarbon, at least one unsaturated hydrocarbon and a molecular oxygen-containing gas to an autothermal cracker wherein they are reacted in the presence of a catalyst capable of supporting combustion beyond the normal fuel rich limit of flammability to provide a hydrocarbon product stream comprising olefins, said process being characterised in that the total hydrocarbon fed to the autothermal cracker comprises at least 20 wt % of unsaturated hydrocarbons.

28. The process according to claim 27, wherein the total hydrocarbon fed to the autothermal cracker comprises at least 10 wt % olefins and at least 10 wt % aromatics.
